(19) United States
(12) Patent Application Publication
(10) Pub. No.: US 2014/0155619 A1
(43) Pub. Date: Jun. 5, 2014

(54) PROCESS FOR PREPARATION OF 1,2,3-TRIAZOLE-4 CARBOXAMIDES

(71) Applicant: TARO PHARMACEUTICAL INDUSTRIES LIMITED, Haifa Bay (IL)

(72) Inventors: Shimon Cherynyak, Haifa (IL); Rosa Cyjon, Haifa Bay (IL); Ilana Ozer, Haifa Bay (IL)

(73) Assignee: TARO PHARMACEUTICAL INDUSTRIES LIMITED, Haifa Bay (IL)

(21) Appl. No.: 14/095,640

(22) Filed: Dec. 3, 2013

Related U.S. Application Data
(60) Provisional application No. 61/732,525, filed on Dec. 3, 2012.

(51) Int. Cl. C07D 249/04

(52) U.S. Cl. CPC C07D 249/04
USPC 548/255

(57) ABSTRACT

The invention relates to the preparation of 1,2,3-triazole-4-carboxamide of general Formula I:

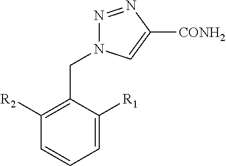

wherein R₁ and R₂ may be the same or different and are selected from H, F, Cl, Br, I, OR, C(O)R, NH₂, NHR or NR₂. R can be a linear or branched alkyl group, for example a C₁-C₄ alkyl group. In exemplary embodiments, R₁ and R₂ are independently selected from H, F, Cl, Br, and I. The compound of Formula I can be Rufinamide.

The invention provides a novel, industrially viable, cost effective process for manufacturing rufinamide and related compounds.

### BACKGROUND OF THE INVENTION

1-(2,6-difluorobenzyl)-1H-1,2,3-triazole-4-carboxamide commonly known as rufinamide is a triazole derivative and is indicated for adjunctive treatment of seizures associated with Lennox-Gastaut syndrome.

Rufinamide was first disclosed in U.S. Pat. No. 4,789,680. The preparation process disclosed in U.S. Pat. No. 4,789,680 involves reacting 2,6-difluorobenzyl chloride and sodium azide in the presence of DMSO to obtain 2,6-difluoro benzyl azide, which is then treated with propiolic acid to give carboxylic acid intermediate which in a further reaction with thionyl chloride gives the corresponding acyl chloride. The acyl chloride intermediate is further reacted with methanolic ammonia to yield rufinamide.

U.S. Pat. No. 6,277,999 discloses a process for the preparation of rufinamide by coupling of 2,6-difluorobenzyl azide and 2-chloroacrylonitrile and partial basic hydrolysis of the intermediate 1-(2,6-difluorobenzyl)-1,2,3-triazolo-4-nitrile into rufinamide.

WO 2010/043849 discloses a process for the preparation of rufinamide by the reaction of 2,6-difluorobenzyl azide with methyl propiolate to obtain methyl 1-(2,6-difluorobenzyl)-1H-1,2,3,-triazole-4-carboxylate, which is then reacted with ammonia to yield rufinamide.

Other multi-step processes for the preparation of rufinamide are disclosed for example in WO 2012/025936, US2010/0234616, and WO9802423. These procedures suffer from various disadvantages which limit applicability on an industrial scale. The prior art processes involved expensive reagents like propiolic acid and 2-chloro acrylonitrile in their processes. The reported processes have complexities over yield, control of impurities during the process and commercial viability.

There is therefore a need for an alternative industrial process which allows preparing rufinamide on a large scale, safely and with improved region-selectivity and higher chemical purity.

### SUMMARY OF THE INVENTION

The present invention provides a novel process for the preparation of pure rufinamide having reduced number of steps and overcoming other disadvantages in the art described above.

It has surprisingly been found that the reaction between benzylazide and 2-haloacrylamide makes the process to obtain rufinamide and structurally related compounds more controllable and reproducible from an industrial point of view. The process also improves overall reaction yields and is more consistent and reproducible. Furthermore, the reaction mixture work-up and the final product recovery require fewer steps, thus affording the final product in short times, higher yields and higher purity.

The invention relates to the preparation of 1,2,3-triazole-4-carboxamide of general Formula I:

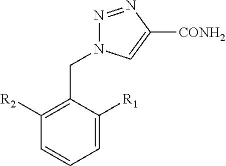

wherein R₁ and R₂ may be the same or different and are selected from H, F, Cl, Br, I, OR, C(O)R, NH₂, NHR or NR₂. R can be a linear or branched alkyl group, for example a C₁-C₄ alkyl group. In exemplary embodiments, R₁ and R₂ are independently selected from H, F, Cl, Br, and I.

In exemplary embodiments, the invention relates to the preparation of rufinamide of Formula II in which both R₁ and R₂ in Formula I are F.

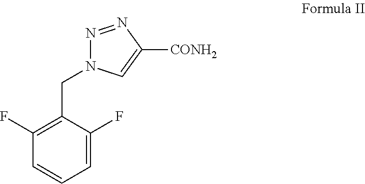 [SMILES: NC(=O)c1cn(Cc2c(F)cccc2F)nn1]

In a preliminary step, a benzyl chloride of general Formula III is converted into a benzylazide of general Formula IV by heating with an azide, for example an alkali metal azide, such as sodium azide. Other leaving groups known in the art that can be displaced by azide, for example Br or I, can be used on the benzyl moiety in place of chloride. In embodiments, the azide may be in excess.

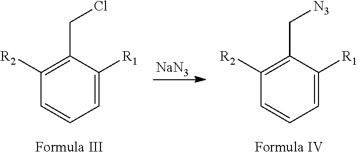

The benzylazide of general Formula IV is then reacted with a 2-substituted acrylamide of Formula V where X is a leaving group such as Cl, Br or I, to directly obtain 1,2,3-triazole-4-carboxamide of general Formula I.

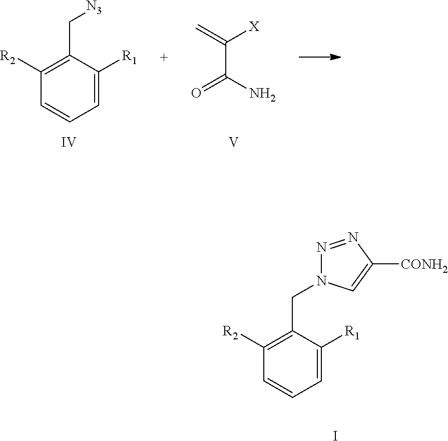

The 1,2,3-triazole-4-carboxamide of general Formula I can be isolated using common methods apparent to persons skilled in the art. In exemplary embodiments, the acrylamide of formula V is 2-chloroacrylamide of Formula VI:

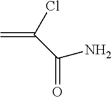

In one exemplary embodiment, the present invention provides a process for the preparation of rufinamide having the structure of Formula II:

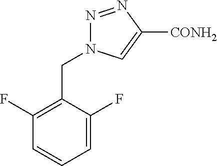 [SMILES: NC(=O)c1cn(Cc2c(F)cccc2F)nn1]

by the steps of
(a) reacting 2,6-difluorobenzyl chloride with sodium azide to obtain 2,6-difluorobenzylazide;

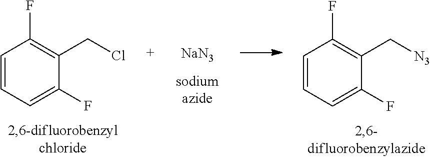

(b) reacting the 2,6-difluorobenzylazide with 2-chloroacrylamide to form rufinamide;

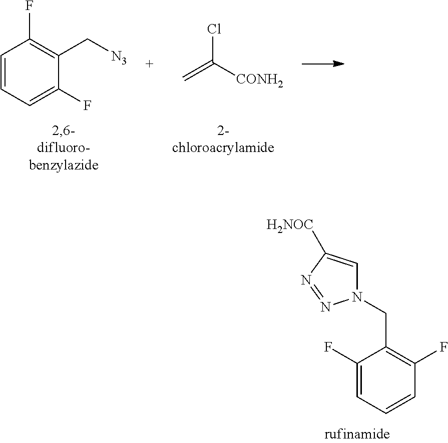

and
(c) isolating the obtained rufinamide.

As will be appreciated, other 2,6-difluorobenzyl starting materials, alkali metal azides and/or 2-haloacrylamides can be used.

In another embodiment, the present invention provides an improved process for the preparation of a 1,2,3-triazole-4-carboxamide of formula I:

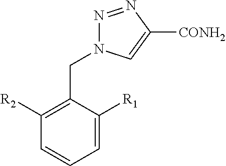

wherein R₁ and R₂ are independently selected from H, F, Cl, Br, I, OR, C(O)R, NH₂, NHR or NR₂, that includes the steps of:
(a) reacting a benzylazide of general Formula IV, with a 2-substiuted acrylamide of general Formula V wherein X is a leaving group such as Cl, Br and I, to obtain the 1,2,3-triazole-4-carboxamide of general Formula I;

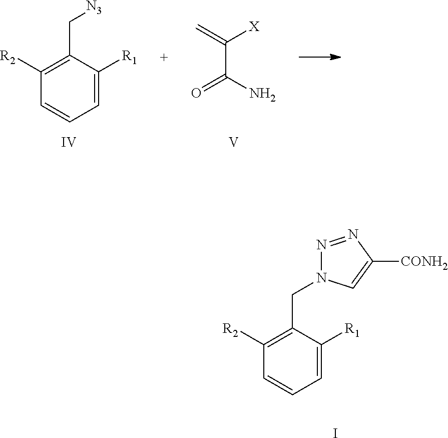

and
(b) isolating the obtained 1,2,3-triazole-4-carboxamide of general Formula I.

In yet another embodiment, the invention relates to the preparation of rufinamide of Formula II in which both R₁ and R₂ in Formula I are F.

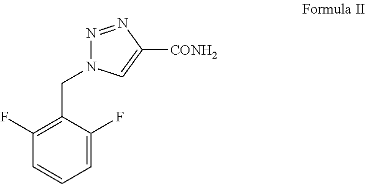 [SMILES: NC(=O)c1cn(Cc2c(F)cccc2F)nn1]

by
(a) reacting 2,6-difluorobenzylazide with 2-chloroacrylamide to form rufinamide;

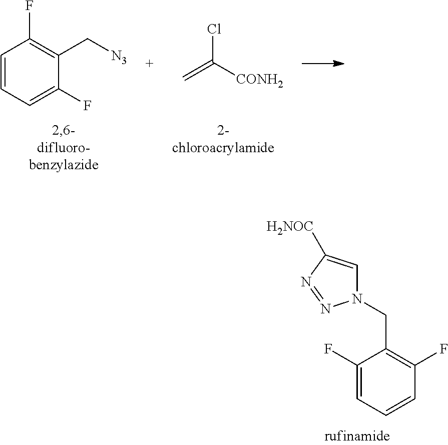

and
(b) isolating the obtained rufinamide.

### DETAILED DESCRIPTION OF THE INVENTION

The present invention relates to the preparation of a 1,2,3-triazole-4-carboxamide of general Formula I:

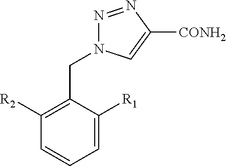

wherein R₁ and R₂ may be the same or different and are selected from H, F, Cl, Br, I, OR, C(O)R, NH₂, NHR or NR₂. In exemplary embodiments, R₁ and R₂ mare independently selected from H, F, Cl, Br, and I. In exemplary embodiments, one or both of R₁ and R₂ are I. In exemplary embodiments, one or both of R₁ and R₂ are F.

In an exemplary embodiment, the invention relates to the preparation of rufinamide having the structure of Formula II in which R₁ and R₂ in Formula I are F.

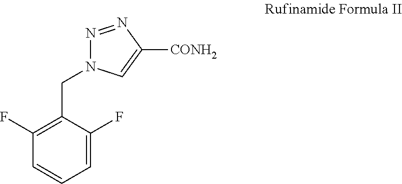 [SMILES: NC(=O)c1cn(Cc2c(F)cccc2F)nn1]

In a preliminary step, a benzyl chloride of general Formula III is converted into a benzylazide of general Formula IV in which R₁ and R₂ are as defined above by heating the benzyl chloride with an excess of an alkali metal azide such as sodium azide or potassium azide. Other leaving groups displaceable by azide, for example Br or I, may be used on the benzyl compound in place of chloride. The reaction can be conducted in, for example, a polar protic solvent, such as water, a C₁-C₄ alcohol such as methanol, isopropanol, butanol, propanol, and mixtures thereof. In embodiments, the reaction can be carried out at a temperature ranging from about 0° C. to the reflux temperature of the reaction mixture. In some embodiments, the equivalent ratio between the benzyl chloride and the alkali metal azide is between about 1:1 to about 1:3. In embodiments, the reaction time may range between about 5 h to about 10 h.

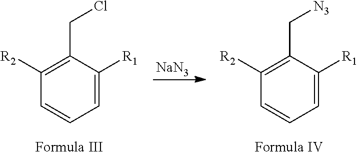

The benzylazide of general Formula IV is reacted with 2-haloacrylamide of general Formula V where X is Cl, Br or I, to obtain a 1,2,3-triazole-4-carboxamide of general Formula I. Other 2-substiuted acrylamides can also be used. The reaction can be conducted in a solvent having a boiling point of at least about 60° C., for example a C₁-C₄ alcohol such as methanol, ethanol, isopropanol, or a mixtures thereof, or in the absence of any solvent. In exemplary embodiments, the reaction is conducted in methanol or ethanol. The reaction can be conducted in the presence of a base such as sodium bicarbonate, potassium carbonate, potassium bicarbonate, potassium tert-butoxide, or triethyl amine. Exemplary embodiments use triethylamine or sodium bicarbonate as the base. In exemplary embodiments, the equivalent ratio between benzylazide and 2-haloacrylamide can be, for example, between about 1:1 to about 1:2.

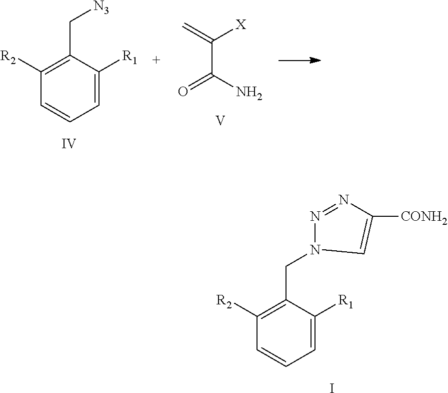

The obtained 1,2,3-triazole-4-carboxamide of general Formula I can be isolated using any suitable method known in the art.

In a more specific embodiment, the invention relates to the preparation of rufinamide, having the structure of Formula II, in which R₁ and R₂ in Formula I are F.

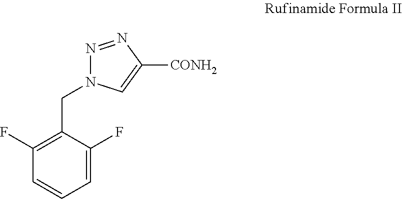 [SMILES: NC(=O)c1cn(Cc2c(F)cccc2F)nn1]

In a preliminary step, 2,6-difluorobenzylazide is obtained by the reaction of 2,6-difluorobenzyl chloride (or other 2,6-difluorobenzyl compound with a suitable leaving group) with an excess of sodium azide (or other suitable azide) as disclosed for example in U.S. Pat. No. 4,789,680, which is incorporated herein by reference. This step is depicted with sodium azide and 2,6-difluorobenzyl chloride in the below scheme:

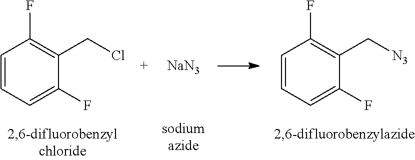 [SMILES: Fc1cccc(F)c1CCl ; [N-]=[N+]=NCc1c(F)cccc1F ; "NaN3"]

The condensation of sodium azide with 2,6-difluorobenzyl chloride can be carried out in a suitable polar protic solvent, for example a solvent selected from the group consisting of a C₁-C₄ alcohol such as methanol, isopropanol, butanol, propanol, and the like, water and mixtures thereof. In exemplary embodiments, the reaction is carried out in water, butanol or mixtures thereof to give 2,6-difluorobenzylazide. The reaction between 2,6-difluorobenzyl chloride and sodium azide can be carried out at a temperature ranging from about 0° C. to the reflux temperature of the reaction mixture. The equivalent ratio between 2,6-difluorobenzyl chloride and sodium azide can be between about 1:1 to about 1:3, for example between about 1:2 to about 1:3. In some embodiments, the reaction time may range between about 5 h to about 10 h.

2,6-Difluorobenzylazide is reacted with, for example, 2-chloroacrylamide to yield rufinamide, as depicted in the below scheme.

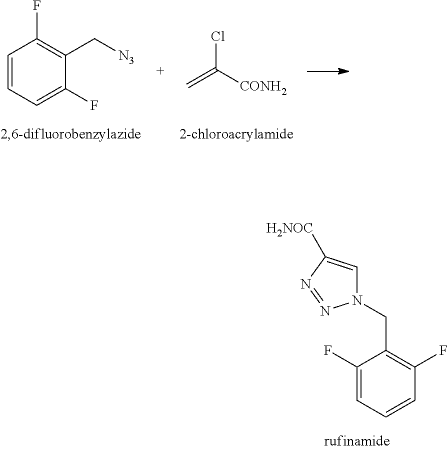

The cyclization reaction can be conducted in the presence of a base such as sodium bicarbonate, potassium carbonate, potassium bicarbonate, or triethylamine. Exemplary embodiments use triethylamine or sodium bicarbonate as the base.

In embodiments, the reaction can be carried out in the absence of solvent, in a C₁-C₄ alcohol solvent, such as methanol, ethanol, isopropanol, in dioxane, or in a mixture of such solvents. In exemplary embodiments, the reaction is conducted in methanol or ethanol. In other embodiments, the cyclization reaction is conducted in dioxane. In alternative embodiments, the cyclization reaction is conducted without any solvent.

The concentration of 2,6-difluorobenzylazide can be, for example, between about 0.1 and about 10 M, such as between about 0.1 and 2M. In exemplary embodiments, the equivalent ratio of 2,6-difluorobenzylazide and 2-chloroacrylamide can be, for example, between about 1:1 to about 1:2.

The reaction between 2,6-difluorobenzylazide and 2-chloroacrylamide can be carried out at a temperature ranging from about 0° C. to the reflux temperature of the solvent in the reaction mixture. In exemplary embodiment, the reaction between 2,6-difluorobenzylazide and 2-chloroacrylamide is carried out at a temperature of at least about 60° C.

The time of reaction depends on the temperature and solvent and may vary between, for example, about 4 h to about 40 h. In exemplary embodiments conducted in the absence of solvent, the second step can be conducted at a temperature of about 120° C. to about 130° C. for about 4 h to 8 h. In exemplary embodiments conducted in ethanol, the temperature of the reaction is between about 75° C. to about 80° C., and the time of reaction is between about 20 h to about 40 h.

In an exemplary method of isolating the product, the suspension formed from the reaction may be cooled and the resultant precipitate filtered, washed with water and dried, for example in a vacuum oven. Other suitable isolation methods apparent to persons skilled in the art may also be used.

In another embodiment, the present invention provides an improved process for the preparation of a 1,2,3-triazole-4-carboxamide of Formula I

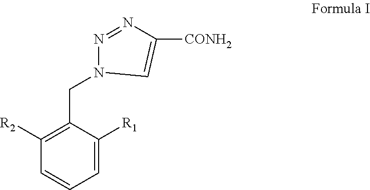

wherein R₁ and R₂ of formula I may be the same or different and are independently selected from H, F, Cl, Br and I. The process can include the steps of:
(a) reacting a benzylazide of general Formula IV wherein R₁ and R₂ are as defined above, with a 2-substituted acrylamide, such as a 2-haloacrylamide of general Formula V wherein X is selected from Cl, Br and I, to obtain the 1,2,3-triazole-4-carboxamide of general Formula I;

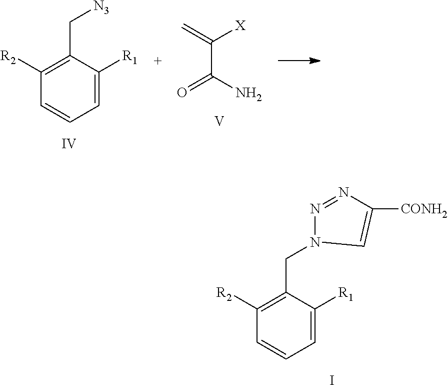

and
(b) isolating the obtained 1,2,3-triazole-4-carboxamide of general Formula I.

Reaction conditions similar or identical to those described below for rufinamide can be used.

The invention also relates to a preparation of rufinamide of Formula II.

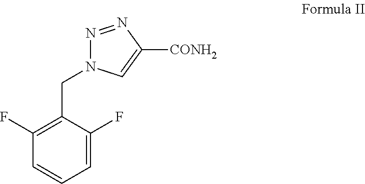 [SMILES: NC(=O)c1cn(Cc2c(F)cccc2F)nn1]

by
(a) reacting 2,6-difluorobenzylazide with 2-chloroacrylamide to form rufinamide;

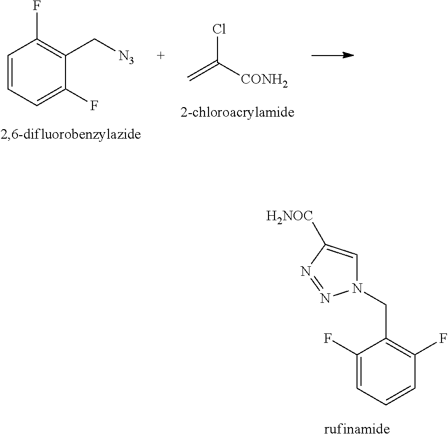

and
(b) isolating the obtained rufinamide.

In exemplary embodiments, the reaction described above between 2,6-difluorobenzylazide and 2-chloroacrylamide to form rufinamide can be conducted in the presence of a base such as sodium bicarbonate, potassium carbonate, potassium bicarbonate, or triethylamine. In exemplary embodiment, the base is triethylamine or sodium bicarbonate.

The reaction can be carried out in a C₁-C₄ alcohol, such as methanol, ethanol, isopropanol, or a mixture thereof, in dioxane or in the absence of solvent. In exemplary embodiments, the reaction is conducted in methanol or ethanol. In other embodiments, the cyclization reaction is conducted in dioxane. In yet another embodiment, the cyclization reaction is conducted without any solvent.

The concentration of 2,6-difluorobenzylazide can be, for example between about 0.1 and about 10 M, preferably between about 0.1 and 2M. In embodiments, the equivalent ratio between 2,6-difluorobenzylazide and 2-chloroacrylamide is between about 1:1 to about 1:2.

The reaction between 2,6-difluorobenzylazide and 2-chloroacrylamide can be carried out at a temperature ranging from about 0° C. to the reflux temperature of the solvent in the reaction mixture. In some embodiments, the reaction between 2,6-difluorobenzylazide and 2-chloroacrylamide is carried out at a temperature of at least about 60° C.

The time of reaction depends on the temperature and solvent utilized. In exemplary embodiments, the reaction time may vary between about 4 h to about 40 h. For example, in exemplary embodiments conducted in the absence of solvent, the cyclization step can be conducted at a temperature of about 120-130° C. for about 4 h to 8 h. In exemplary embodiments in which the reaction with 2-haloacrylamide is conducted with ethanol as a solvent, the temperature of the reaction can be between about 75° C. to about 80° C., and the time of reaction can be between about 20 h to about 40 h.

In an exemplary method of isolating the product, the suspension formed in the reaction may be cooled and the result precipitate filtered, washed with water and dried, for example in a vacuum oven. Other suitable isolation methods apparent to persons skilled in the art may also be used.

The size of rufinamide crystals obtained according to the present invention is characterized by a D₅₀ value ranging from 25 μm to 250 μm. In embodiments, D₉₀ is 100 μm or less, 40 μm or less, or 20 μm or less. In embodiments, D₉₀ can be at least 5 μm or at least 10 μm. In embodiments, D₉₀ can range from 5 to 100 μm, from 5 to 40 μm, from 10 to 40 μm, from 5 to 20 μm or from 10 to 20 μm. If desired, particle size value can be reduced by micronisation or fine grinding.

The rufinamide product as obtainable by the process of the present invention has chemical purity equal to or higher than about 90.0%, about 95.0% or about 99.0% (as determined by HPLC). In exemplary embodiments, the chemical purity is equal to or higher than about 99.9%, which is a quality suitable to meet the regulatory requirements for medicinal products.

### EXAMPLES

### Example 1

### Benzyl Azide Preparation

2,6-Difluorobenzyl Azide—36 g of sodium azide (550 mmoles) was dissolved in 200 ml water. 20 g (120 mmoles) of 2,6-difluorobenzyl chloride was added and the mixture was heated to 65-75° C. and stirred at this temperature for 5 hrs. The formed emulsion was cooled and the layers were allowed to separate. The aqueous layer was discarded and the obtained oil (19.7 g, 95%) that contained up to 99% of 2,6-difluorobenzylazide was used for the next step.

Other benzyl azides were prepared by analogous way from sodium azide and the correspondent substituted Benzyl Chlorides:

Benzyl Azide—99% yield; IR spectrum (KBr): 2100 cm⁻¹;

2-Fluorobenzyl Azide—66% yield; IR spectrum (KBr):2109 cm⁻¹; and

2-Chloro-6-fluorobenzyl Azide-61% yield; IR spectrum (KBr): 2098 cm⁻¹.

### Example 2

### 1,2,3-Triazole-4-carboxamides preparation

Rufinamide—A mixture of 22 g (126 mmoles) of 2,6-difluorobeznylazide and 20 g (189 mmoles) of 2-chloroacrylamide was dissolved in 110 ml of absolute ethanol. 15 g (20.3 ml, 147 mmoles) of triethylamine was added and the mixture was heated to reflux and stirred at this temperature for about 24 hrs. The formed suspension was cooled and the white precipitate was filtered and washed with additional amount of ethanol. The obtained rufinamide was dried in vacuum oven at 100-110° C. until a constant weight was obtained. Yield 22 g (76%). Purity 99.8%.

8.547(1H, H-5), 7.813 (1H,NH), 7.578-7.477 (1H, H-4), 7.227-7.161 (2H, H-3,H-5), 7.452 (1H,NH), 5.732 (2H, CH2);

¹³C-NMR-spectrum (DMSO-d₆): 162.439, 162.341, 159.135, 159.037(C-6′, C-2′, F-splitting), 161.227 (C=0), 142.477 (C-4), 131.886, 131.747, 131.609 (C-4′, F-splitting), 126.673 (C-5), 112.043, 111.950, 111.821, 111.720 (C-3′,C-5′, F-splitting), 111.345, 111.003, 110.662 (C-1′, F-splitting), 41.138 (CH₂);

Mass-spectrum: 239 (MH⁺), 127 [MH-112]⁺.

The obtained Rufinamide is identical to USP standard.

Other 1,2,3-triazole-4-carboxamides were prepared by analogous way form corresponding Benzyl Azides and 2-Chloroacrylamide:

1-Benzyl-1H-1,2,3-triazole-4-carboxamide—Yield: 81%.—IR spectrum (KBr): 3408(NH), 1643(CO), 1556(C═C); MS: 203 (MH⁺).

1-(2-Chloro-6-fluorobenzyl)-1H-1,2,3-triazole-4-carboxamide—Yield: 85%. IR spectrum (KBr): 3402(NH), 1630(CO), 1608, 1598(C═C, C═N); ¹H-NMR spectrum (DMSO-d₆): 8.546 (1H, H-5), 7.839 (1H,NH), 7.50-7.63 (1H, H-4′), 7.498 (1H, NH), 7.40-7.45 (1H,H-5′), 7.349 (1H,H-3′), 5.776 (2H, CH₂); ¹³C-NMR-spectrum (DMSO-d₆): 162.130, 160.470(C-6′, C-2′, F-splitting), 161.275 (C=0), 142.639 (C-4), 134.885, 134.854 (C-2′, F-splitting), 131.963, 131.898 (C-4′, F-splitting), 126.857 (C-5), 125.897, 125.867 (C3′, F-splitting), 120.706, 120.590 (c-1′, F-splitting), 115.06, 114.914 (c5′, F-splitting), 44.670 (CH₂); MS: 255.0 (MH⁺), 143.2[MH-112]+.

1-(2-Fluorobenzyl)-1H-1,2,3-triazole-4-carboxamide—Yield: 80% IR spectrum: 3396 (NH), 1653 (CO), 1617,1589 (C═C,C═N); MS:221 (MH⁺).

### Example 3

### Rufinamide preparation

A mixture of 22 g (126 mmoles) of 2,6-difluorobeznylazide and 20 g (189 mmoles) of 2-chloroacrylamide was dissolved in 110 ml of absolute ethanol. 11 g (130 mmoles) of sodium bicarbonate was added and the mixture was heated to reflux and stirred at this temperature for 30 hrs. The formed suspension was cooled, diluted with water and the result precipitate was filtered, washed with 50 ml water and dried under vacuum at 100-110° C. Yield 23 g (74%).

### Example 4

### Rufinamide preparation

A mixture of 22 g (126 mmoles) of 2,6-difluorobeznylazide and 20 g (189 mmoles) of 2-chloroacrylamide was dissolved in 110 ml of dioxane and heated to 80-90° C. and stirred at this temperature for 24 hrs. The obtained suspension was cooled to RT and rufinamide was filtered, washed with dioxane and dried under vacuum at 100-110° C. Yield 24 g (82%). Purity 99.0%.

### Example 5

### Rufinamide preparation

A mixture of 22 g (126 mmoles) of 2,6-difluorobeznylazide and 20 g (189 mmoles) of 2-chloroacrylamide was heated to 120-130° C. and stirred at this temperature for 5-6 hrs. The reaction mixture was cooled to 60-70° C. and diluted with ethanol. The obtained precipitate was filtered, washed with ethanol and dried under vacuum at 100-110° C. Yield 65%. Purity 97%.

1. A process for the preparation of a 1,2,3-triazole-4-carboxamide of formula I 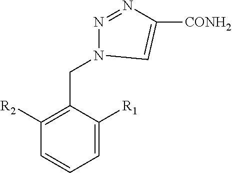
comprising:
(a) reacting a benzyl chloride of general formula III with an azide to obtain a benzylazide of general formula IV, wherein R₁ and R₂ are as defined above; 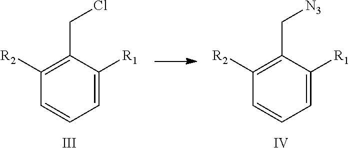
(b) reacting the benzylazide of general Formula IV with a 2-halideacrylamide of general Formula V, wherein X is selected from Cl, Br and I, to obtain the 1,2,3-triazole-4-carboxamide of general Formula I; 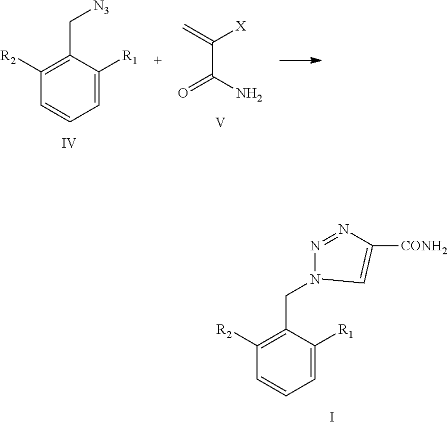
and
(c) isolating the obtained 1,2,3-triazole-4-carboxamide of general Formula I
wherein R₁ and R₂ are independently selected from the group consisting of H, F, Cl, Br, I, OR, C(O)R, NH₂, NHR or NR₂, wherein R is a linear or branched alkyl group.

2. The process according to claim 1, wherein both R₁ and R₂ are F.

3. The process according to claim 1, wherein the azide is an alkali metal azide.

4. The process according to claim 3, wherein the alkali metal azide is sodium azide or potassium azide.

5. The process according to claim 1, wherein the 2-halideacrylamide is 2-chloroacrylamide.

6. The process according to claim 1, wherein reaction of step (i) is carried out in the presence of a polar protic solvent.

7. The process according to claim 6, wherein the polar protic solvent is C₁-C₄ alkanol, water, or mixtures thereof.

8. The process according to claim 1, wherein reaction step (ii) is carried out in the presence of a solvent having boiling point of at least about 60° C., or in the absence of any solvent.

9. The process according to claim 8, wherein reaction step (ii) is carried out in the presence of C₁-C₄ alkanol or dioxane.

10. The process according to claim 1, wherein reaction step (ii) is conducted in the presence of a base selected from sodium bicarbonate, potassium carbonate, potassium bicarbonate, potassium tert- butoxide and triethylamine.

11. A process for the preparation of rufinamide of Formula II, 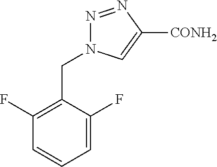 [SMILES: NC(=O)c1cn(Cc2c(F)cccc2F)nn1]
comprising:
(a) reacting 2,6-difluorobenzyl chloride with sodium azide to obtain 2,6-difluorobenzylazide; 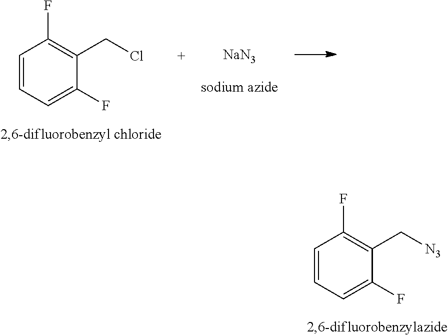 [SMILES: Fc1cccc(F)c1CCl ; [N-]=[N+]=NCc1c(F)cccc1F ; "NaN3"]
(b) reacting 2,6-difluorobenzylazide with 2-chloroacrylamide to form rufinamide; 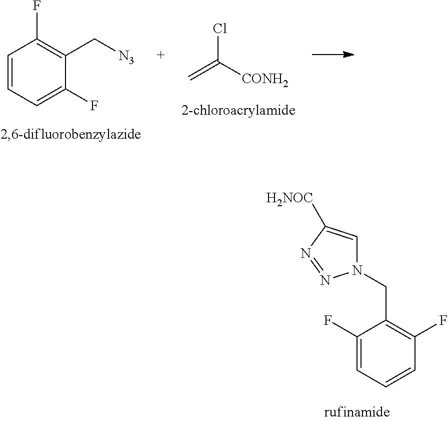
and
isolating the obtained rufinamide.

12. The process according to claim 11, wherein reaction of step (i) is carried out in the presence of water.

13. The process according to claim 11, wherein reaction step (ii) is carried out in the presence of ethanol or dioxane as a solvent, or in the absence of any solvent.

14. The process according to claim 11, wherein the reaction between 2,6-difluorobenzyl chloride and sodium azide is performed at a temperature between 0° C. to the reflux temperature of the solvent in the reaction mixture.

15. The process according to claim 12, wherein the reaction between 2,6-difluorobenzylazide and 2-chloroacrylamide is carried out at a temperature ranging from about 0° C. to the reflux temperature of the solvent in the reaction mixture.

16. The process according to claim 11, wherein the equivalent ratio between the 2,6-difluorobenzyl chloride and the sodium azide is between about 1:1 to about 1:3.

17. A process for the preparation of a 1,2,3-triazole-4-carboxamide of formula I 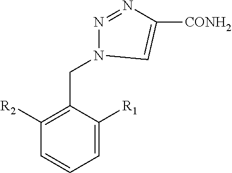
wherein R₁ and R₂ are independently selected from H, F, Cl, Br and I, comprising:
reacting a benzylazide of general Formula IV wherein R₁ and R₂ are independently selected from H, F, Cl, Br and I, with a 2-halideacrylamide of general Formula V wherein X is selected from Cl, Br and I, to obtain the 1,2,3-triazole-4-carboxamide of general Formula I; 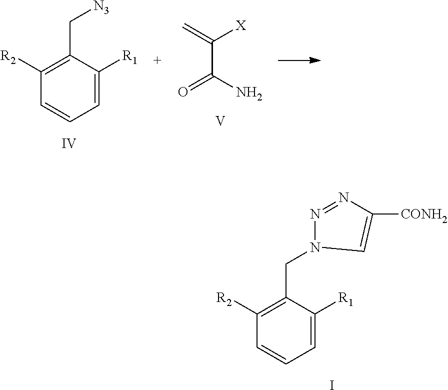
and
isolating the obtained 1,2,3-triazole-4-carboxamide of general Formula I;
wherein R₁ and R₂ are independently selected from the group consisting of H, F, Cl, Br, I, OR, C(O)R, NH₂, NHR or NR₂, wherein R is a linear or branched alkyl group.

18. The process according to claim 17, wherein both R₁ and R₂ are F.

19. The process according to claim 17, wherein the 2-halideacrylamide is 2-chloroacrylamide.

20. The process according to claim 17, wherein reaction step (i) is carried out in the presence of a solvent having boiling point of at least about 60° C., or in the absence of any solvent.

21. The process according to claim 20, wherein reaction step (i) is carried out in the presence of C₁-C₄ alkanol or dioxane.

22. The process according to claim 17, wherein reaction step (i) is conducted in the presence of a base selected from sodium bicarbonate, potassium carbonate, potassium bicarbonate, potassium tert-butoxide and triethylamine.
